(19) United States
(12) Patent Application Publication
(10) Pub. No.: US 2024/0326041 A1
(43) Pub. Date: Oct. 3, 2024

(54) MICROFLUIDIC INDUCTION OF RESEARCH BASED NEURAL INJURY

(71) Applicant: Worcester Polytechnic Institute, Westborough, MA (US)

(72) Inventors: Hamilton A. White, Worcester, MA (US); Dirk R. Albrecht, Chatham, NJ (US)

(21) Appl. No.: 18/621,550

(22) Filed: Mar. 29, 2024

Related U.S. Application Data
(60) Provisional application No. 63/528,543, filed on Jul. 24, 2023.
(60) Provisional application No. 63/461,749, filed on Apr. 25, 2023.
(60) Provisional application No. 63/455,295, filed on Mar. 29, 2023.

(51) Int. Cl. B01L 3/00

(52) U.S. Cl. CPC B01L 3/5027

(57) ABSTRACT

A microfluidic device for evaluation of test subjects for induced neural injury performs testing of multiple test subjects based on uniform and repeatable test stimuli for evaluating neural response for research including traumatic brain injury. A microfluidic device contains multiple test subjects and delivers a consistent, measured test stimuli simulating TBI to each of the test subjects simultaneously. The result is a system to assess neural function, behavior, and neural structure of small animals responsive to sonication-induced traumatic brain injury, to investigate risk and potential recovery. The microfluidic device disposes test subjects at a uniform distance from an injury inducing surface that emits sonication energy to simulate TBI. The uniform distance ensures that each test subject receives the same, controlled injury stimuli, and the test subjects may be evaluated with an attached microscope or video input, or may be extracted from the microfluidic device for further evaluation.

### RELATED APPLICATIONS

This patent application claims the benefit under 35 U.S.C. § 119 (e) of U.S. Provisional Patent App. No. 63/455,295, filed Mar. 29, 2023, entitled “MICROFLUIDIC INDUCTION OF RESEARCH BASED NEURAL INJURY,” U.S. Provisional Patent App. No. 63/461,749, filed Apr. 25, 2023, entitled “INHIBITION AND RECOVERY FROM NEURAL INJURY,” and U.S. Provisional Patent App. No. 63/528,543, filed Jul. 24, 2023, entitled “NEUROLOGICAL MODEL FOR TRAUMATIC INJURY,” all incorporated herein by reference in entirety.

### BACKGROUND

Neurological research presents challenges for injury evaluation while a test subject is still living. Current Traumatic Brain Injury (TBI) models typically use mammalian animals (e.g. rodents, pigs, monkeys) and blunt force impacts or blast waves and assess behavioral and cognitive effects. These are relatively low throughput (one to a few animals tested at a time), show large variations among populations, and are difficult to use as a system for post injury amelioration investigation. Conventional systems may use bead disruptors and surface wave acoustic systems to deliver injury, but are still limited in scalability by the number of test subjects available.

### SUMMARY

A microfluidic device for evaluation of test subjects for induced neural injury performs testing of multiple test subjects based on uniform and repeatable test stimuli for evaluating neural response for research of traumatic brain injury. A microfluidic device contains multiple test subjects and delivers a consistent, measured test stimuli simulating TBI to each of the test subjects simultaneously. The result is a system to assess neural function, behavior, and neural structure of small animals before, during and after sonication-induced traumatic brain injury, to investigate risk factors and potential therapies enhancing recovery. The microfluidic device disposes test subjects at a uniform distance from an injury inducing surface that emits sonication energy to simulate TBI in the test subjects. The uniform distance ensures that each test subject receives the same, controlled injury stimuli, and the test subjects may be evaluated with an attached microscope or video input, or may be extracted from the microfluidic device for further evaluation.

Configurations herein are based, in part, on the observation that test subjects for TBI are often selected from animals based on an ability to simulate injury and a similarity to human physiology for ensuring meaningful results. Bioethical and cost considerations are also important factors. Unfortunately, conventional approaches to TBI and similar neural trauma research suffer from the shortcoming ensuring that each test subject receives the same, controlled injury stimuli. Injury inducement may be inconsistently applied and cause varied levels of injurious injury to be received across multiple test subjects.

Accordingly, configurations herein substantially overcome the shortcomings of conventional approaches by delivering a consistent and repeatable injury stimuli to multiple test subjects in the microfluidic device by applying sonication (ultrasound) and ensuring that each test subject is located within a consistent distance from a sonication energy emitting surface in the microfluidic containment. The result is a system to create consistent traumatic injury in a large number of animals simultaneously, allowing assessment of genetic and pharmacological post-injury interventions in a screening-compatible system. The disclosed approach facilitates academic research by providing a method of assessing neural, behavior and structural responses to traumatic neural injury before, during and after the injury event. Conventional approaches do not allow assessment of responses at the cellular and subcellular level.

Current TBI models typically use mammalian models (e.g. rodents) and blunt force impacts (e.g. falling weights) and assess behavioral and cognitive effects. These are relatively low throughput (few animals tested), show large variations among populations, and are difficult to assess on a cellular level to uncover underlying mechanisms. Configurations herein demonstrate bath sonication of the nematode C. elegans to overcome such limitations. Animals in the disclosed system are consistently and repeatably injured at controllable levels ranging from no effect, minor injury, major trauma and death. The disclosed methods are compatible with live in vivo monitoring of neuronal structures by fluorescence microscopy, neural activity and stimulated responses by calcium imaging, and behavioral quantification by machine vision, in dozens to tens of thousands of animals at once. Bath sonication is particularly advantageous because the system can accept microfluidic devices and hydrogel encapsulation methods for assessment of responses, which enables longitudinal studies in the same individual animals before, during, and after injury to see how the injury affects brain function acutely and chronically up to days later.

In further detail, configurations herein provide testing and observation device for simulation of traumatic injury testing of laboratory subject, including a test containment adapted for containing a plurality of test subjects, and an injury inducement medium operable for inducing an injury of similar magnitude to each of a plurality of test subjects in the test containment. An actuation circuit is configured for energizing the injury inducement medium for a predetermined interval selected based on a target injury stimuli directed at each of the plurality of test subjects.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a microfluidic inducement apparatus configured for use with the disclosed approach;

FIG. 2 is a schematic diagram of a microfluidic device as in FIG. 1;

FIG. 3 shows test subjects contained in the microfluidic device as in FIG. 2;

FIG. 4 shows a process flow of a use case with the microfluidic device as in FIG. 2;

FIG. 5 shows a timeline of data gathered from the process of FIG. 4;

FIGS. 6A and 6B show injury classification based on the process of FIGS. 4 and 5;

FIGS. 7A and 7B show a neural structure in a test subject responsive to the device of FIG. 2;

FIGS. 8A-8C show results of test substances for treatment of an injury induced by the microfluidic device of FIGS. 1-4; and

FIG. 9 shows a diagram of therapeutic results from the test substances as in FIGS. 8A-8C.

### DETAILED DESCRIPTION

In configurations depicted below, example configurations of a microfluidic device for research-based injury inducement is disclosed in conjunction with test subjects such as Caenorhabditis elegans (C. elegans), a small, wormlike animal with remarkable similarity to a human neural structure. The disclosed approach is suited to other small animals for research-based injury inducement of multiple small animals in a containment by applying a consistent level on a injury inducement medium to the test subjects.

An estimated international yearly incidence of 295 patients per 100,000 yearly have mild-TBI (mTBI). Symptoms in this injured population can greatly reduce quality of life. TBI can affect sensorimotor functions persistently over time; deleterious sensorimotor impacts on function and behavior are closely linked with changes in the sensory processing of information and place a significant burden on patient and their caretakers. Previous work has highlighted that after single and repeat head injury (RHI), reduced olfaction was a key indicative sequelae, and has been associated with short and long-term deficits in memory. Other common symptoms of head injury include increased touch sensitivity, which was conserved across species from mice to humans and in some cases is persistent. With long growth times and ethical considerations in their handling for experimentation, murine models commonly used for TBI studies are not conducive to high throughput, and repeatable study.

Additionally, studying TBI in animal model systems is made more difficult by the many complex interrelationships that make up the so-called post-trauma injury cascade. An ideal animal model for studying the injury process would share a strong degree of neurochemical homology with humans but allow rapid growth cycles and high-throughput genetic analysis of large number of individuals that underwent the same levels of repeatable traumatic injury. Caenorhabditis elegans (C. elegans) is a transparent, isogenic, rapidly growing nematode (roundworm) that has significant genetic homology, high degrees of genetic control by novel and classical techniques, and the capacity for high-throughput study. Establishing and linking the implications of neural injury in both C. elegans and humans will help verify and validate the use of nematodes in under-standing brain injury, and the exploration and translation of new ideas of novel mechanisms for reducing the burden of head injury.

Indications for use of a particular animal model for studying the TBI process are not well defined in conventional approaches, with observed results having variance in metanalysis. Instruments of injury that mimic neurotrauma and are quantifiable, repeatable and reproducible, with outcomes that correlate with the mechanisms of injury being deemed as most clinically relevant. Configurations herein consider that TBI is injury to the brain or central processing region of an organism, having either transient or prolonged sensorineural, behavioral or morphological defects of the nervous system.

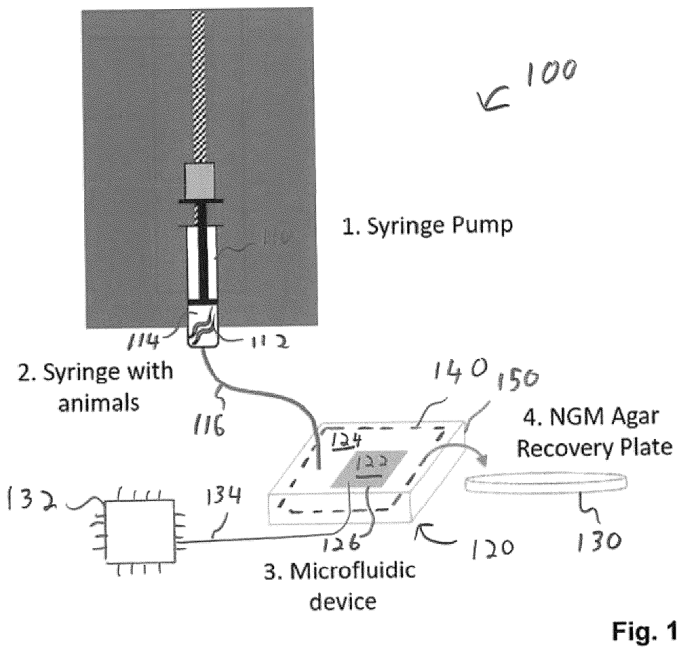

FIG. 1 is a context diagram of a microfluidic inducement apparatus configured for use with the disclosed approach. Referring to FIG. 1, in a sonication environment 100, a syringe pump 110 introduces a sample 112 of test subjects in a buffer fluid 114 into a microfluidic device 120. A fluidic coupling 116 introduces the test subjects 116 and buffer fluid 114 into the device 120. The microfluidic device 120 uses a bath sonicator 150 to encapsulate a test containment 124 with an injury inducement medium 126 such as a sonication element 122. The fluidic aspect provides both a mobility environment for the test subjects (c. elegans in the disclosed example) and a propagation medium for the injury inducement medium 126, sonication energy in the disclosed approach.

To injure animals, a continuous flow of buffer fluid is pumped through a microfluidic device 120 having serpentine microchannels in a bath sonicator-induced cavitation field. Animals are trapped in the flow and directed through the cavitation field. Exposure time is determined by channel length and flow velocity. The disclosed device can create multiple injury magnitudes by parallel channels of different length and/or flow velocity, resulting in different sonication exposure times. Animals are recovered on an agar plate 130 for further transfer and analysis using widefield or high-resolution microscopy, or encapsulated in hydrogel and assessed over time in presence of various pharmacological agents.

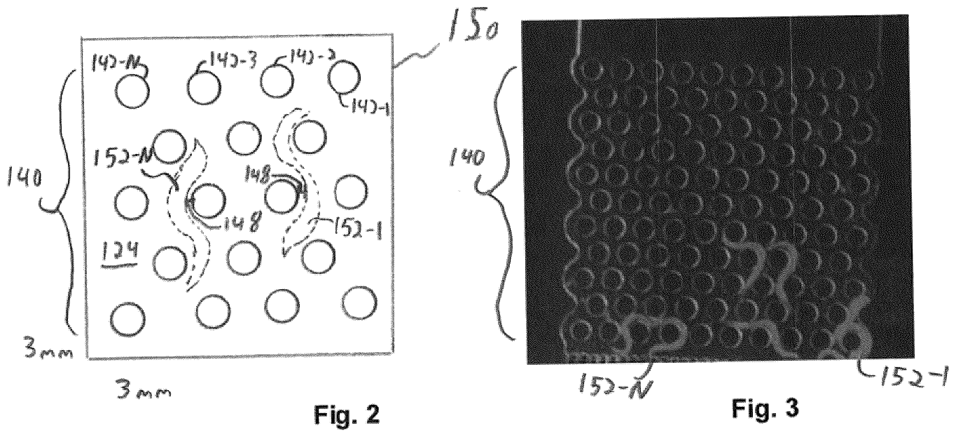

FIG. 2 is a schematic diagram of a microfluidic device 120 as in FIG. 1. Referring to FIGS. 1 and 2, the disclosed testing and observation system and device 120 for simulation of traumatic injury testing of laboratory subjects includes the test containment 124 adapted for containing a plurality of test subjects. In the test containment 124, a post array 140 has a plurality of pylons 142-1 . . . 142-N (142 generally). The pylons 142 form a plurality of surfaces in an arrangement for disposing each of the plurality of test subjects within a predetermined distance from one or more of the surfaces. In the example configuration, the test containment 124 is a bath sonicator and the injury inducement medium 126 is provided in the form of sonication energy from a sonication element 122. The injury inducement medium delivers a target injury stimuli defined by a duration and a frequency of sonication energy emitted from each of a plurality of surfaces of the test containment 124. Each pylon 142 is therefore responsive to the injury inducement medium 126 for emitting an injury signal.

The post array 140 and plurality of surfaces defined by the pylons 142 is particular amenable to the wormlike, serpentine form factor of the C. elegans test subjects. By sizing and spacing the pylons 142 based on an average width and length of the C. elegans, the pylons form a consistent spacing 144 between them from a uniform diameter 146 of each of the pylons. Once disposed in the post array 140, each test subject 152-1 . . . 152-N (152 generally) positions around and between the pylons 142 such that a minimum distance 148 is maintained for each of the test subjects 152. The post array 140 effectively sets an average distance between the plurality of surfaces in the test containment based on a size of each of the plurality of test subjects 152, such that each test subject is within a minimum distance from at least one of the plurality of surfaces. By ensuring each test subject is within the minimum distance, an effective and consistent exposure to the sonication energy emitted from the surfaces of the test containment 124.

The microfluidic device 120 includes the control circuit 132 and an interface 134 operable for generating an actuation signal for inducing injury. The actuation signal is typically defined in terms of a duration and a periodic interval, such that the injury inducement medium 126 responds to the interface for actuation of the sonication element 122 based on the duration and periodic interval. The injury inducement medium 126 is therefore a sonication emitter configured for emitting sonication energy from each of a plurality of surfaces on the pylons 142 and walls/floors in the test containment 124. The injury inducement medium 126 is therefore operable for inducing an injury of similar magnitude to each of a plurality of test subjects 152 in the test containment 124, resulting from each of the test subjects 152 being disposed within the predetermined distance 148 from a surface emanating the injury inducement medium 126. By introducing a plurality of test subjects 152 in the test containment 124, the injury inducement medium 126 is configured to create a reproducible injury across each of test subjects.

FIG. 3 shows a photograph of test subjects 152 contained in the microfluidic device 120 as in FIG. 2. Referring to FIGS. 2 and 3, it can be observed how the post array 140 locates the pylons 142 such that the C. elegans attains a serpentine form and “wraps” around the pylons to maintain the minimum distance 148. Since a typical experiment depicted herein utilizes young adult animals, the post array 140 where the animals reside is made of 200 μm diameter pylons 142 spaced 100 μm apart in a 3×3 mm matrix using a hexagonal pattern. The height of the post array 140 is generally 55-70 μm given the need to prevent overlapping of animals in the device. 40 μm spaced barriers on either end of the post array allow free-flow of stimulus fluids and buffer, but prevent animals escaping. A minimal distance of each test subject 152 to the nearest surface (typically a post 142) is between within 50 μm, typically between 5-15 μm and preferably not more than 10 μm from the nearest surface to the test subject 142. Other suitable dimensions may be employed.

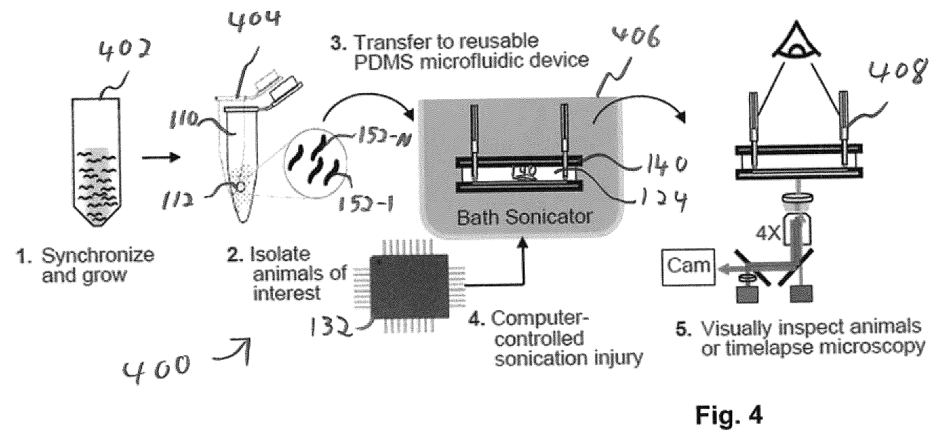

FIG. 4 shows a process flow 400 of a use case with the microfluidic device as in FIG. 2. Referring to FIGS. 1-4, at step 402, the test subjects 152 are grown or produced in sufficient quantity and at low cost compared to, for example, rodent specimens. A batch of test subjects is isolated and introduced via the buffer fluid 114 and suitable coupling 116, as depicted at step 404. The microfluidic device 120 including the post array 140 receives the test subjects 152-N, as shown at step 406. Computer-controlled bath sonication induces titratable cavitation conditions within a microfluidic post-array 140. The actuation circuit 132 is configured for energizing the injury inducement medium 126 for a predetermined interval selected based on a target injury stimuli directed at each of the plurality of test subjects 152. Using the microfluidic array, the control circuit 132 defines activation duration and iteration of the injury inducement medium 126.

The test containment 124 further comprises a visualization medium and a recordation medium, as depicted at step 408. The visualization medium is configured for rendering an observable reaction by each of the plurality of test subjects to the injury inducement medium and the recordation medium stores an indication of an interval of response of each of the plurality of test subjects to the injury inducement medium.

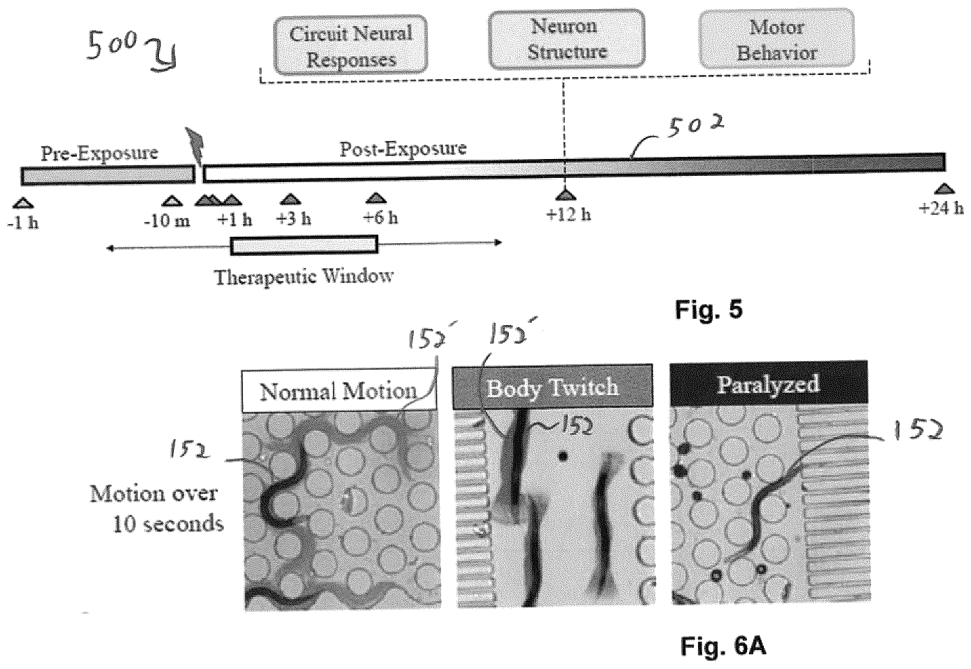

FIG. 5 shows a timeline 500 of data gathered from the process of FIG. 4. Referring to FIGS. 4 and 5, direct examination in the post array 140 or transfer via agar plate 130 allow quantification of neural and structural outcomes using both widefield epifluorescent and spinning disk confocal microscopy. Analyses, perturbations and therapeutic interventions can be made across timescales 502: before, soon-after and long and after injury. In other words, post-trauma degradation attributable to the induced injury can be effectively tracked across each of the test subjects, for example observing 1, 3 and 6 hour increments following injury.

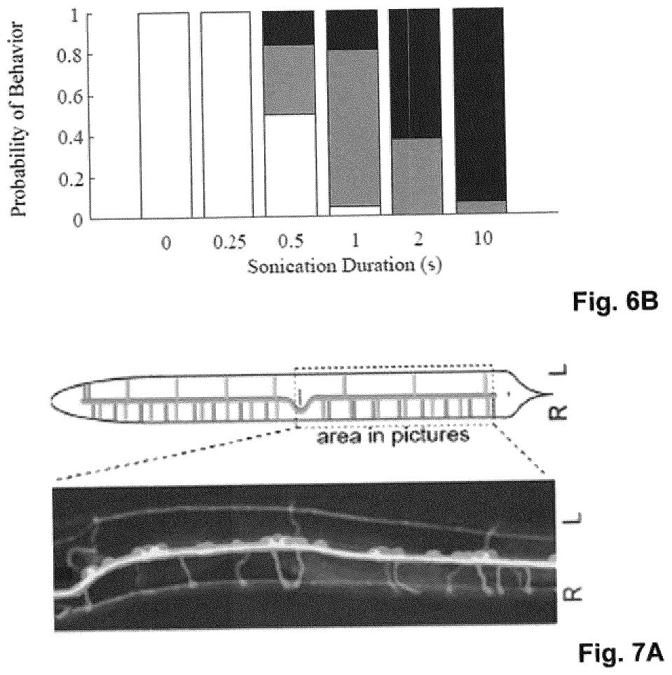

FIGS. 6A and 6B show injury classification based on the process of FIGS. 4 and 5. Referring to FIGS. 2 and 6A-6B, objectives of the disclosed microfluidic device 120 include 1) a geometry for consistent inducement of injury; 2) analysis of a single neuron; and 3) observation before and for specified durations after injury, across the range of test subjects 152. FIG. 6A shows states of observable injury, based on movement of subjects 152 to a subsequent position 152′. Normal motion allows observation of the subject 152 to a subsequent position 152′ around a different set of pylons 142. An injury resulting in only twitching movement shows a generally non-motive shift to a subsequent position 152, and a complete paralysis shows no deviation of a position of the test subject 152. FIG. 6B shows by a shaded histogram the proportion or probability (vertical axis) of the twitching or paralysis behavior following specified time increments along the horizontal axis. As a general benchmark, animals in the microfluidic arena were checked visually to ensure proper movement, then injured using the automated sonication injury system. Upon removal, animals may be assessed visually for behavioral state according to the following definitions:
Paralysis: movement phenotype characterized by no obvious gross bodily movement.
Twitch: movement phenotype characterized by shaking or convulsions of the body or a substantial part of the main body of the animal. Animals in this phenotype do not show ability to coordinate their bodily motion in the normal sinusoidal or thrashing movement pattern, and have “jerky” fractional movement of the head, tail, or both simultaneously.
Normal: animals can coordinate their bodily movement and smoothly move around the microfluidic arena freely (although movement may at times be “slow”).

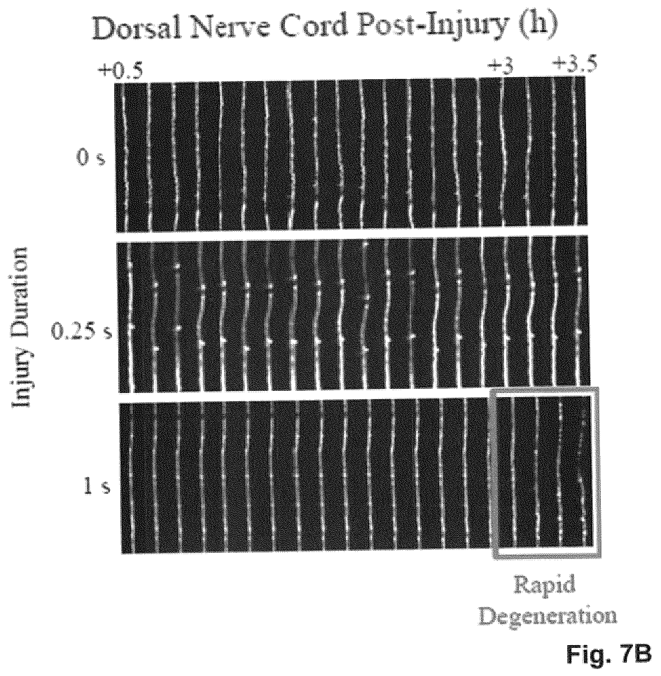

FIGS. 7A and 7B show a neural structure in a test subject responsive to the device of FIG. 2. Referring to FIGS. 2 and 7A-7B, via fluorescently labelled neurons of the test subject(s) 142 in FIG. 7A, the motor nerve cords that drive locomotion and behavior can be visualized. It was observed that 0-0.25 second injury durations do not cause nerve cord degeneration. However, moderate injury durations (˜1 second) cause progressive degeneration which rapidly accelerates from t=3-3.5 hours post injury, shown in FIG. 7B. The top and middle rows of FIG. 7B show little to no neuron compromise, however the bottom row, depicting 1 second duration of injury stimuli, show not only short term degradation, but cumulative degradation around 3-3.5 hours (horizontal axis) to the portion of rapid degeneration. Injury durations >2 seconds cause systemic fracture of the dorsal nerve cord immediately after injury, with full degeneration of the ventral nerve cord by t=3 hours.

The disclosed microfluidic array which allows rapid assessment of multiple test subjects 152 with uniform application of the injury inducement medium 126 also allows similar testing efficiency for remedial testing. The disclosed approach addresses a clinical and research issue in academic and clinical medicine by providing a method that allows recovery of neural activity, and in particular chemosensory neural activity, after injury via methods that without treatment cause loss of neural activity and body degeneration. The composition of matter described below includes a widely available antioxidant and potent/specific calcium channel blocker to prevent the types of post-injury activation that can lead to neurodegeneration and failure of biological recovery.

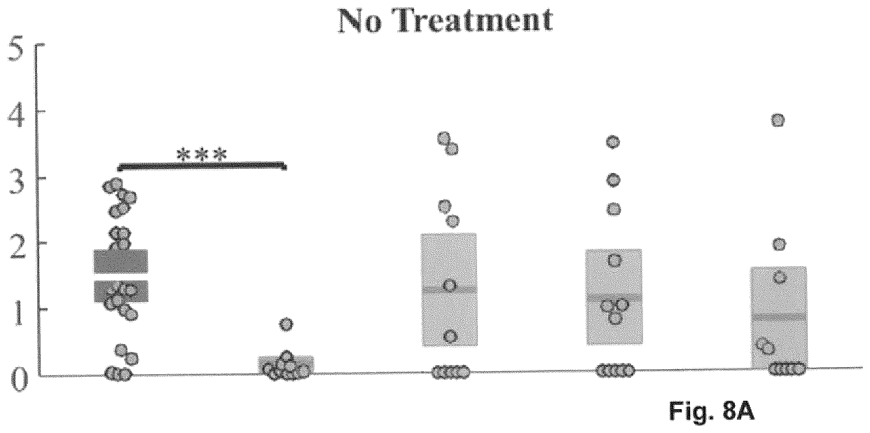

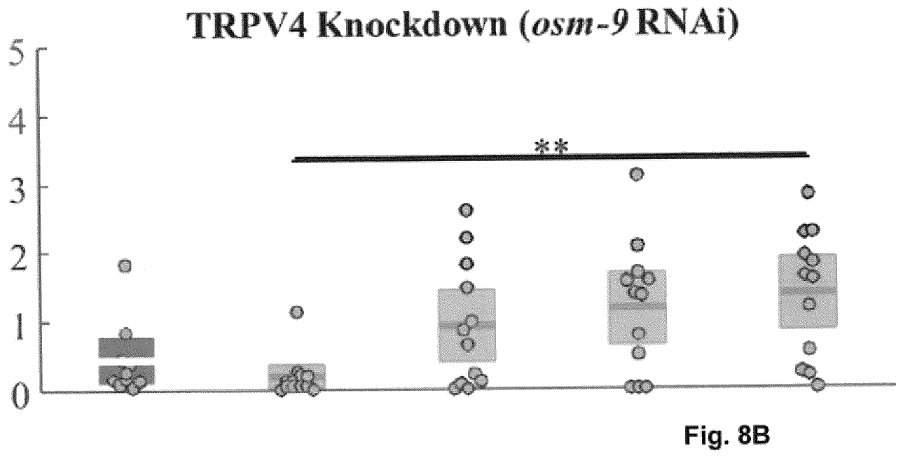

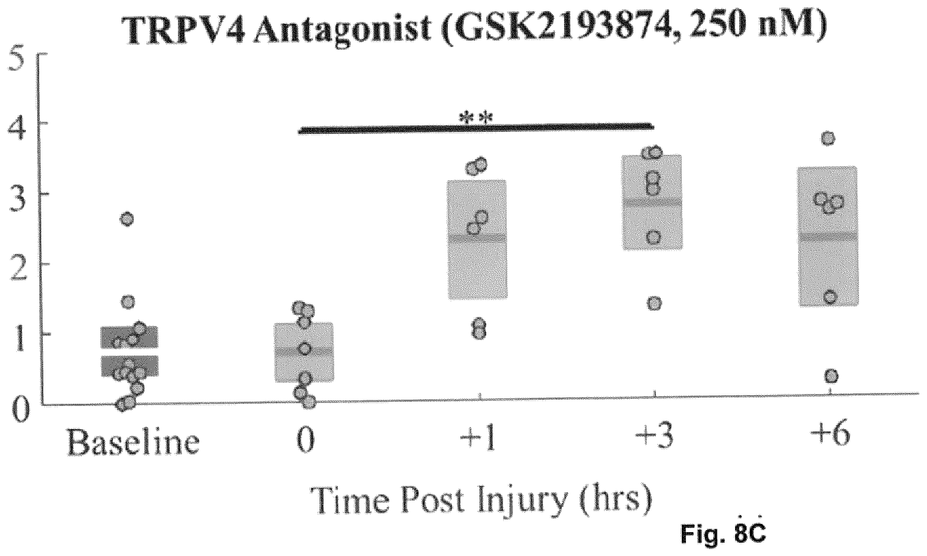

FIGS. 8A-8C show results of test substances for treatment of an injury induced by the microfluidic device of FIGS. 1-4. Referring to FIGS. 8A-8C, one approach to therapeutic and/or prophylactic neural treatment includes two chemical agents that are diluted to physiological concentrations and administered in liquid form to the test animals using their food. Drug compositions are applied for a series of several hours to allow the biological function of the composition to manifest. Animals are removed from drug administration and allowed to immobilize under anesthetic for a period of ˜60 minutes in a microfluidic device before being injured. Referring to FIGS. 8A-8C, neural activity is assessed immediately before and repeatedly for a period of time after the injury is induced (horizontal axis), where the horizontal axis shows AWA dF/F0, mean ±SEM.

The described action of the drug composition is to inhibit the ability of calcium influx channels to operate for a period of time before and during the injury, allowing later recovery of the neuron response when the action of the drug composition ceases.

### Drug Composition

N-acetyl-L-cysteine: a potent antioxidant with neuroprotective properties (here we use a concentration near adverse effects concentration due to lack of inherent bioavailability, other compositions including using N-acetyl-L-cysteine amide would likely work better at lower usage concentrations).
TRPV4 antagonists: potent and specific TRPV4-channel blockers (here we use GSK2193874 at 250nM administered concentration, but believe other like drugs would work via similar mechanisms). GSK2193874 is a commercially available, orally active, potent, and selective TRPV4 antagonist with ICsos of 2 nM and 40 nM for rTRPV4 and hTRPV4.
   Currently there are no known FDA approved drugs targeting amelioration of neural defects after TBI.

Since the disclosed results are consistently seeing near-100% of post-treatment test animals recover some level of neural activity (many near full, normal response levels), configurations indicate that this solution is at least 0.5-5× better than existing uses of NAC for neuroprotection in TBI and TRPV4 is a worthwhile pharmacological target for potential treatment development.

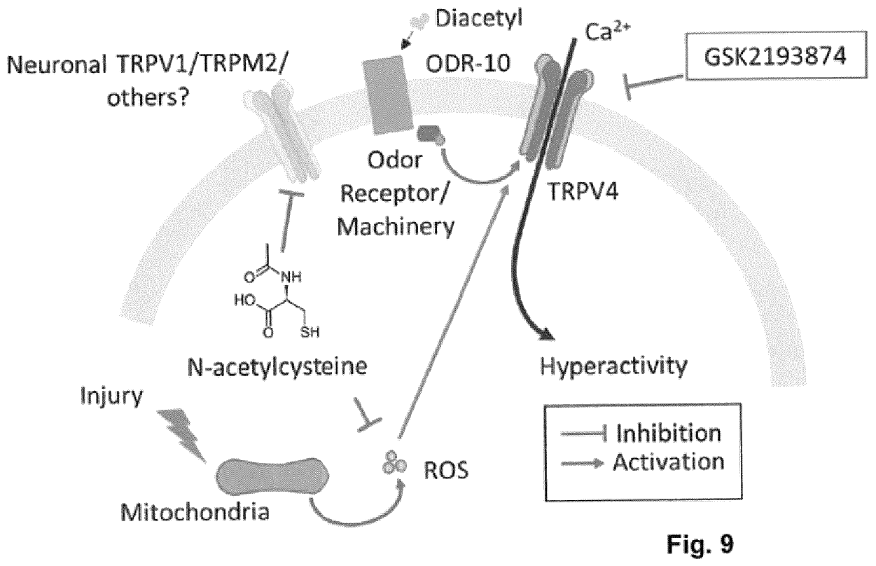

FIG. 9 shows a diagram of therapeutic results from the test substances as in FIGS. 8A-8C. Referring to FIGS. 8A-9, in a particular example, N-acetylcysteine (NAC) shows preclinical evidence of improving outcomes after TBI via its antioxidant properties. Initial behavioral data indicates that 2 hour pre-exposure to 1 mM NAC lessens behavioral vulnerability, and NAC-induced recovery produces calcium transients similar to ‘osm’ mutants in wild-type C. elegans. Untreated animals do not show significant recovery due to response variability.

A small-scale RNAi screen identified osm-9: knockdown before injury allowed recovery +6 hrs. after (n=21, n=11 trackable post-injury). The introduction of osm-9/rRPV4 via an orthogonal method (mammalian TRPV4 antagonists) confirmed initial finding of neural recovery (n=14, n=6 trackable post-injury). This demonstrates a pharmacological composition of multiple neuroactive agents including a potent intracellular antioxidant and potent TRPV4 channel inhibitor allowing recovery from controlled traumatic neural injury in a plurality of test specimens when applied prophylactically for research purposes.

N-acetylcysteine (NAC) shows pre-clinical evidence of improving outcomes after TBI via its antioxidant properties. Initial behavioral data suggests that 2 hour pre-exposure to 1 mM NAC lessens behavioral vulnerability and NAC-induced recovery produces calcium transients similar to ‘osm’ mutants in wild-type C. elegans (hTRPV4 ortholog6). A small-scale RNAi screen identified osm-9: knockdown before injury allowed recovery +6 hrs after (n=21, n=11 trackable post-injury) osm-9/TRPV4 via an orthogonal method (mammalian TRPV4 antagonists) confirmed initial finding of neural recovery (n=14, n=6 trackable post-injury).

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A testing and observation device for simulation of traumatic injury testing of laboratory subjects, comprising:
a test containment adapted for containing a plurality of test subjects;
an injury inducement medium operable for inducing an injury of similar magnitude to each of a plurality of test subjects in the test containment; and
an actuation circuit configured for energizing the injury inducement medium for a predetermined interval selected based on a target injury stimuli directed at each of the plurality of test subjects.

2. The device of claim 1 wherein the injury inducement medium is a sonication emitter configured for emitting sonication energy from each of a plurality of surfaces in the test containment.

3. The device of claim 1 wherein the test containment includes a plurality of surfaces in an arrangement for disposing each of the plurality of test subjects within a predetermined distance from a surface of the plurality of surfaces.

4. The device of claim 3 wherein the plurality of surfaces includes a post array having a plurality of pylons, each pylon responsive to the injury inducement medium for emitting an injury signal.

5. The device of claim 1 wherein the injury of similar magnitude is based on each of the test subjects disposed within a predetermined distance from a surface emanating the injury inducement medium.

6. The device of claim 1 wherein the injury inducement medium includes sonication energy, and the target injury stimuli is defined by a duration and a frequency of sonication energy emitted from each of a plurality of surfaces of the test containment.

7. The device of claim 1 further comprising a plurality of test subjects disposed in the test containment, the injury inducement medium configured to create a reproducible injury across a plurality of test subjects.

8. The device of claim 7 wherein an average distance between the plurality of surfaces in the test containment is selected based on a size of each of the plurality of test subjects, such that each test subject is within a minimum distance from at least one of the plurality of surfaces.

9. The device of claim 1 wherein the test containment further comprises a visualization medium and a recordation medium, the visualization medium configured for rendering an observable reaction by each of the plurality of test subjects to the injury inducement medium and the recordation medium stores an indication of an interval of response of each of the plurality of test subjects to the injury inducement medium.

10. The device of claim 1 further comprising a control circuit and an interface, the control circuit operable for generating an actuation signal, the actuation signal having a duration and a periodic interval, the injury inducement medium responsive to the interface for actuation based on the duration and iteration based on the periodic interval.

11. The device of claim 4 wherein the post array has an area of between 1-5 mm×1-5 mm and a spacing of between 50-150 μm between the pylons.

12. The device of claim 11 wherein the test subjects are nematodes having a size for achieving a distance between 0-15 μm from the pylons.

13. A method for testing and treatment of neurological injury, comprising:
disposing a plurality of test subjects in a test containment;
providing an injury inducement medium operable for inducing an injury of similar magnitude to each of a plurality of test subjects in the test containment; and
energizing the injury inducement medium for a predetermined interval selected based on a target injury stimuli directed at each of the plurality of test subjects.

14. The method of claim 13 wherein the injury inducement medium is a sonication emitter configured for emitting sonication energy from each of a plurality of surfaces in the test containment.

15. The method of claim 13 further comprising arranging a plurality of surfaces in the test containment for disposing each of the plurality of test subjects within a predetermined distance from a surface of the plurality of surfaces. 16 The method of claim 15 further comprising disposing each of the test subjects within a predetermined distance from at least one of the plurality of surfaces emanating the injury inducement medium, thereby imposing injury of similar magnitude to each of the test subjects.

17. The method of claim 13 further comprising emitting sonication energy for a predetermined duration and a frequency from each of a plurality of surfaces of the test containment.

18. The method of claim 13 further comprising generating an actuation signal from a control circuit, the actuation signal having a duration and a periodic interval, the injury inducement medium responsive to an interface to the control circuit for actuation based on the duration and iteration based on the periodic interval.

19. The method of claim 13 further comprising:
introducing a chemical agent for inhibiting the ability of calcium influx channels in the test subjects for mitigating an effect of the injury inducement medium; and
observing the test subjects for assessing the degree of mitigation.

20. The method of claim 19 wherein the chemical agent includes a TRPV4 antagonist.
